(19) United States
(12) Patent Application Publication
(10) Pub. No.: US 2024/0374691 A1
(43) Pub. Date: Nov. 14, 2024

(54) GLP-1R AGONISTS FOR USE IN A TREATMENT OF NEUROLOGIAL IMPAIRMENT ASSOCIATED WITH VIRAL INFECTION

(71) Applicant: Neuraly, Inc., Gaithersburg, MD (US)

(72) Inventors: Adam C. Bell, Monrovia, MD (US); Viktor Roschke, Bethesda, MD (US); Subbu Karanth, Germantown, MD (US); Seulki Lee, Ellicott City, MD (US)

(21) Appl. No.: 18/686,711

(22) PCT Filed: Aug. 25, 2022
(86) PCT No.: PCT/US2022/075451

§ 371 Date: Feb. 26, 2024

Related U.S. Application Data
(60) Provisional application No. 63/237,059, filed on Aug. 25, 2021.

(51) Int. Cl. A61K 38/26; A61K 38/38; A61K 47/60; A61P 25/28

(52) U.S. Cl. CPC A61K 38/26; A61K 38/38; A61K 47/60; A61P 25/28

(57) ABSTRACT

Long-acting glucagon like peptide 1 receptor agonists (GLP-1R agonists) reduce and inhibit pathological processes caused by activated microglia that give rise to long-term neurological impairment resulting from infection with a virus such as COVID-19. A preferred long-acting GLP-1R agonist is PEGylated exenatide, administered systemically, preferably subcutaneously. The compositions are particularly suited for treating, alleviating, and/or preventing one or more neurological symptoms associated with microglial activation elicited by a virus such as COVID, for example, cognitive impairment.

### CROSS-REFERENCE TO RELATED APPLICATION

This application claims the benefit of and priority to U.S. Ser. No. 63/237,059 filed Aug. 25, 2021, and which is incorporated by reference in its entirety.

### FIELD OF THE INVENTION

The invention is generally in the field of treatment for neurological disorders, and in particular, methods and compositions for selectively treating chronic cognitive impairment associated with viral infection.

### BACKGROUND OF THE INVENTION

The COVID-19 (coronavirus disease 2019) pandemic caused by severe acute respiratory syndrome coronavirus 2 (SARS-COV-2) has to date resulted in over 500 million documented COVID cases worldwide. Neurological symptoms are emerging as relatively common sequelae, with persistent cognitive impairment affecting approximately one in four COVID survivors. While more common in individuals who had experienced severe COVID requiring hospitalization, even those with mild symptoms in the acute phase may experience lasting cognitive dysfunction. Colloquially known as “COVID fog” or “Brain Fog”, this syndrome of COVID-associated cognitive impairment is characterized by impaired attention, concentration, speed of information processing, memory, and executive function. Together with increased rates of anxiety, depression, disordered sleep, and fatigue, this syndrome of cognitive impairment contributes substantially to the morbidity of “long COVID” and in many cases prevents people from returning to their previous level of occupational function. Neurologic consequences associated with “Brain Fog” are evident as a persistent impairment in sustained attention in approximately 80% of patients suffering from “long COVID”. Given the scale of SARS-COV-2 infection, this syndrome of persistent cognitive impairment represents a major public health crisis.

The symptoms of “long-haul” COVID can be debilitating and there are no approved treatments. In a recent National Institutes of Health study of 3,700 COVID “Long Haulers” in 56 countries, nearly half could not work full-time for six months after getting sick. While more than half of these respondents say they never sought hospital care, more than 2,400 reported symptoms such as fatigue, shortness of breath, trouble sleeping, “brain fog”, cognitive dysfunction, or memory loss persisting six months or longer. With long-haul symptoms potentially impacting millions of people, NIH has announced a $1B fund to study the disease (Collins F. S., (2021 Feb. 23) NIH launches new initiative to study “Long COVID”, National Institute of Health, retrieved from www.nih.gov/about-nih/who-we-are/nih-director/statements/nih-launches-new-initiative-study-long-covid#:˜:text=1%20write%20to%20announce%20a,period%20of%20a%20few%20weeks.).

Given that the adverse effect of microglial reactivity in the CNS outlasts the direct damaging effect of viral infection (Thakur K T et al., Brain. 2021 Apr. 15: awab148), the SARS-COV-2 pandemic represents a global public health emergency that warrants proactive research to establish the implications of microglia to avert potential incidence of neuropsychiatric disorders and neurodegenerative diseases, which could be pervasive in the coming years as a result of the growing numbers of cases, survivors and re-current waves. The neurological impairment or “Brain Fog”, found in long-haul COVID-19 sufferers includes symptoms like poor concentration and poor memory that are similar to mild cognitive impairment seen early in Alzheimer's disease, but in many cases, patients recover, indicating the underlying mechanism may differ. Both AD and PD are driven by chronic stimulation of microglia-dependent inflammation process, which ultimately leads to death of brain-resident neurons. The regional specificity of neuronal loss is driven by the presence of protein aggregates thought to be key drivers of microglial activation (beta-amyloid in the case of Alzheimer's disease and alpha-synuclein in the case of Parkinson's disease). COVID-19 infection is known to worsen Alzheimer's disease (Xia, et al., Transl Neurodegener. 2021 Apr. 30; 10 (1): 15) and in Parkinson's disease 75% of those infected experienced new or worsening PD-related symptoms (Brown, et al., J Parkinsons Dis. 2020; 10 (4): 1365-1377).

Under inflammatory conditions such as CNS viral infection, microglia promote innate and adaptive immune responses to protect the host. However, during viral infection, dysregulated microglia can cause neurocognitive impairment (Chhatbar et al., Cell Mol Immunol. 2021 February; 18 (2): 250-258 (2021)). This dysregulation is the target of GLP-1R agonist therapy for long-haul symptoms associated with coronavirus (aka COVID-19 infection), but numerous other viruses cause brain inflammation leading to neurologic complications. While microglia are clearly involved in neurodegeneration, they are also thought to play a positive role in adult memory function (Augusto-Oliveira M et al., Cell. 2019 Oct. 22; 8 (10): 1293). In a study of individuals living with HIV, when microglial activation was probed using PET-based translocator-protein ligands (TSPO), cognition was inversely associated with microglial activation (Rubin L H et al., AIDS. 2018 Jul. 31; 32 (12): 1661-1667). Microglial contact with neurons can enhance activity, but not when microglia are activated (Akiyoshi R et al., eNeuro. 25; 5 (5) (2018)), illustrating one way that microglial activation might lead to altered neuronal function independent of neuronal cell death.

Data suggest that COVID-19 infection causes microglial activation in some patients. In mice, microglial activation develops approximately 1 week after infection. Following mild respiratory COVID in mice, persistently impaired hippocampal neurogenesis, decreased oligodendrocytes, and myelin loss were evident together with elevated CSF cytokines/chemokines including CCL11 (Fernandez-Castaneda, et al., Cell, Vol. 185, pages 2452-2468, Kao and Frankland, Cell, Vol. 185, pages 2391-2393, doi: 10.1016/j.cell.2022.06.020). The current theory is that the effects of COVID on microglia are indirect, the source activator is unknown. Regardless, persistent (or sub chronic) microglial activation leads to neuron dysfunction in some individuals. In some individuals, symptoms resolve without treatment. In others they persist. Regardless, the consequences can be serious. The Mayo Clinic reported on an evaluation of the first 100 patients who entered their post-COVID program (Vanichkachorn G et al., Mayo Clinic Proceedings, 96, 7, 2021, 1782-1791). Eighty of the 100 patients reported fatigue while 59 reported breathing problems and 59 reported neurological symptoms ranging from headaches to dizziness. Forty-five reported cognitive impairment. Only 30% had returned to full-time work. The average age of the patients was 45, and the majority did not have underlying health problems before infection. Fatigue was reported to be a major symptom. Hospital centers focused on treatment of patients with long-haul covid are all over the USA, including clinics at Johns Hopkins, George Washington, Medstar-DC, and UMD-Baltimore.

Accordingly, new treatments for modifying the effects of long-term neurological impairment associated with COVID-19 are needed.

Therefore, it is an object of the invention to provide compositions that reduce or prevent the pathological processes associated with the development and progression of chronic neurological disease associated with COVID-19, and methods of making and using thereof.

It is also an object of the invention to provide compositions and methods for treating neurologic symptoms, reducing, alleviating, or preventing impairments associated with COVID-19.

It is a further object of the invention to provide compositions and methods for reducing pathological microglial activation associated with COVID-19.

### SUMMARY OF THE INVENTION

It has been established that long-acting glucagon like peptide 1 receptor agonists (long-acting GLP-1R agonists) reduce and/or inhibit pathological processes such as microglial activation that give rise to chronic disease symptoms such as neurologic complications secondary to viral infection. Other GLP-1R agonists share the same target receptor and should have the same effects on microglia, though their clinical efficacy may not be the same and may differ depending on tissue distribution, blood brain barrier access, receptor kinetics, and exposure at well-tolerated doses.

Under inflammatory conditions such as CNS viral infection, microglia promote innate and adaptive immune responses to protect the host. However, during viral infection, dysregulated microglia can cause neurocognitive impairment (Chhatbar et al., 2021 Cell Mol Immunol. 2021 February; 18 (2): 250-258). Numerous other viruses also cause brain inflammation leading to neurological complications.

Methods of treating or preventing COVID-related and other virus-related neurologic dysfunction in a subject are provided. The methods include administering to a subject suffering from or at risk of suffering from COVID complications a pharmaceutically effective amount of a composition including a long-acting GLP-1R agonist to prevent or alleviate one or more symptom of COVID in the subject. The GLP-1R agonist therapy targets the dysregulation of microglia described above for the treatment long-haul symptoms associated with a coronavirus (e.g., COVID-19) infection and other virus-related neurological dysfunction. Exemplary long-acting GLP-1R agonists include a PEGylated GLP-1R agonist analog, a Fc fusion GLP-1 analog, an albumin fusion GLP-1 analog, or derivatives thereof. A preferred long-acting GLP-1R agonist is a PEGylated exenatide, or a PEGylated exenatide analog. Short acting GLP-1R agonists may also be effective if administered more frequently.

In some embodiments, the amount of a long-acting GLP-1R agonist is effective to inhibit the secretion of inflammatory and/or neurotoxic mediators secreted from activated innate immune cells. Typically, the innate immune cells are microglia and/or astrocytes. In particular embodiments, the long-acting GLP-1r agonist is administered in an effective amount to reduce inflammatory or neurotoxic mediators selected from the group including of TNF-α, IL-1α, IL-1β, IFN-γ, IL-6, and C1q as compared to an appropriate control. In other embodiments, the effective amount of the long-acting GLP-1r agonist reduces the cell populations of activated microglia and reactive astrocytes in the subject.

The methods treat, reduce, or prevent one or more symptom arising from or after COVID infection, include fatigue, cognitive difficulties, deficits in attention and executive function, headache, numbness/tingling, dizziness, insomnia, depression, and anxiety-related symptoms. Exemplary cognitive difficulties that are alleviated or prevented by the GLP-1r agonists include impairment of one or more functions selected from reasoning, problem solving, spatial planning and target detection, confusion, attention deficit and reduced executive function. Those who suffer from Brain Fog often report a lack of focus or mental clarity, as if being in a “haze” or “fog.” Symptoms include confusion and disorientation, which can result in an inability to recall facts, find the right words to complete sentences, or generally collect one's thoughts in a constructive and coherent manner. This type of cognitive impairment can also cause memory problems and excessive tiredness, creating a mental slowdown and inability to complete simple assignments. Typically, the extent of the cognitive difficulties in the subject is determined by comparison to an age-matched control and with age-adjusted standard rating scales. For example, Cambridge neuropsychological test automated battery (CANTAB) is a set of tests that measure various aspects of memory, attention, reaction time, executive functioning, and decision making, and found that CFS subjects exhibited deficits in working memory and impaired attention. Paced Auditory Serial Addition Test (PASAT) and the Digit Span Test are other examples of tests that may be employed to rate the cognitive functioning. A comprehensive list of tests for potential use in the assessment of “brain fog” was developed originally for the assessment of cognitive deficits associated with chronic fatigue syndrome (Ocon A J Front Physiol. 2013 Apr. 5; 4:63). Cognitive testing may also include assessments of executive function and attention deficit measured using a variety of validated testing systems include the Adult ADHD Self-Report Scale, the Adult ADHD Clinical Diagnostic Scale (ACDS), the Brown Attention-Deficit Disorder Symptom Assessment Scale (BADDS) and the Adult ADHD Rating Scale-IV (ADHD-RS-IV). An exemplary control is the same cognitive function in the same subject prior to having COVID-19. Treatment effect is measured as test score at baseline (prior to treatment) and after treatment. A placebo control group is also used to assess normal variation in the study population that is not attributable to the study treatment.

In some embodiments, the symptoms of COVID include impaired motor, memory, and cognitive skills. Therefore, in some embodiments, the methods restore motor, memory, and cognitive skills in the subject. For example, in some embodiments, the subject's motor, memory, and cognitive skills are restored at least in part to those prior to having COVID-19. In other embodiments, the symptoms of COVID include one or more neurological symptoms selected from low energy, problems concentrating, disorientation and difficulty finding the right words. Therefore, in some embodiments, the methods treat or prevent one or more neurological symptoms selected from low energy, problems concentrating, disorientation and difficulty finding the right words in the subject. In some embodiments, the subject has no other symptoms associated with COVID-19. In some embodiments, the subject was infected with a virus that causes COVID-19 one, two, three, four, five, six, seven, eight, none, ten or more than ten days, weeks, or months prior to the onset of one or more long-term symptoms of COVID.

In some embodiments, treatment is once-weekly injection for 12 weeks, or until symptoms resolve. Treatment is resumed if symptoms reoccur.

Subjects with pre-existing neurologic conditions that affect memory, including Alzheimer's disease and Parkinson's disease, may be more susceptible. Other pre-existing conditions may predispose patients to increased risk of long COVID or of more severe symptoms. Pre-existing conditions that may make the subject more likely to acquire brain fog are neurodegenerative diseases, cerebrovascular disease, diabetes, ADHD, and chronic fatigue syndrome.

In some embodiments, the composition is administered via oral administration, intravenous administration, parenteral administration, intramuscular administration, or subcutaneous administration. In some embodiments, the composition is administered in pills, capsules, tablets, or liquids.

In particular embodiments, the composition is administered to the subject between one and four times in a month, inclusive. In some embodiments, the composition is administered regularly, at an interval selected from once a week, every two weeks, approximately once a month, once every two months and once every three months. In other embodiments, the composition is administered between one and three times every 6 months, or only once, inclusive. In some embodiments, the composition has a half-life in vivo of 12±8 days in humans. In some embodiments, the composition is administered to a human subject at a dose of between 0.001 mg/kg and 100 mg/kg, inclusive. In a particular embodiment, the composition is administered to a human subject at a dose between 0.1 mg and 10 mg, inclusive. In a particular embodiment, the composition is administered to a human subject at a dose of 5 mg. For example, in some embodiments, the composition is administered at a dose of 5 mg once a week for up to 6 months. Typically, the composition is administered to the subject in two or more doses, over a period of between one and 10 days, weeks, or months, inclusive.

Methods for treating cognitive impairment associated with COVID in a subject including systemically administering to the subject an effective amount of a PEGylated exenatide, or a PEGylated exenatide analog to treat, alleviate, reverse and/or prevent one or more aspects of the cognitive impairment in the subject are also described. Typically, the PEGylated exenatide, or a PEGylated exenatide analog is administered in an effective amount to decrease activated microglia in the brain of the subject, improve cognition, or combinations thereof. In preferred embodiments, the PEGylated exenatide, or a PEGylated exenatide analog is administered in a dose of between 0.1 mg and 20 mg, inclusive.

### BRIEF DESCRIPTION OF THE DRAWINGS

FIGS. 1A-1D are bar graphs showing amounts of pro-inflammatory cytokines in activated/non-activated microglia cells, treated with PBS (white solid/black solid), or NLY01 (white spotted/black spotted), respectively, showing amounts of C1q (0-80 ng/m) (0-80 ng/ml) (FIG. 1A); IL1α (0-800 pg/ml) (FIG. 1B); TNF-α (0-400 pg/ml) (FIG. 1C); and IL1β (0-600 pg/ml) (FIG. 1D), respectively.

FIGS. 2A and 2B are bar graphs showing the effect of NLY01 in activated/non-activated microglia in the brain, treated with PBS (white solid/black solid), or NLY01 (white spotted/black spotted), respectively. FIG. 2A shows relative IBa-1 intensity (1-4); and FIG. 2B shows microglia density (0-150 cells/mm²), respectively.

FIGS. 3A-3E are bar graphs showing Fold Induction of C1q (FIG. 3A); of TNF (FIG. 3B); of IL1-α (FIG. 3C); of IL1-β) (FIG. 3D); and of IL-6 (FIG. 3E), respectively, treated with PBS (white solid/black solid), or NLY01 (white spotted/black spotted), respectively.

### DETAILED DESCRIPTION OF THE INVENTION

### I. Definitions

As used herein, “activated microglial cells” refers to microglia, the resident immune cells of the CNS, which normally respond to neuronal damage and remove the damaged cells by phagocytosis. Under steady-state conditions, microglia are maintained in a “resting” state through interactions with cell surface and soluble factors from surrounding cells. Microglia become activated following exposure to pathogen-associated molecular patterns (PAMPs) and/or endogenous damage-associated molecular patterns (DAMPs), and removal of the immune-suppressive signals. Activated microglia can acquire different phenotypes depending on cues in its surrounding environment.

Neuroinflammation, defined as inflammation of nervous tissue, is initiated in response to a variety of endogenous and exogenous sources including invading pathogens, neuronal injury, and toxic compounds. It is characterized by glial cell activation, the release of inflammatory molecules, increased blood-brain barrier permeability, and recruitment of peripheral immune cells into the brain. The chronic activation of microglia may in turn cause neuronal damage through the release of potentially cytotoxic molecules such as proinflammatory cytokines, reactive oxygen intermediates, proteinases and complement proteins.

The term “therapeutic agent” refers to an agent that can be administered to treat one or more symptoms of a disease or disorder. The term “prophylactic agent” generally refers to an agent that can be administered to prevent disease or to prevent certain conditions, such as a vaccine.

The term “pharmaceutically acceptable salt”, as used herein, refers to derivatives of the compounds defined herein, wherein the parent compound is modified by making acid or base salts thereof. Example of pharmaceutically acceptable salts include, but are not limited to, mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The term “therapeutically effective amount” refers to an amount of the therapeutic agent that produces some desired effect at a reasonable benefit/risk ratio applicable to any medical treatment. The effective amount may vary depending on such factors as the disease or condition being treated, the size of the subject, or the severity of the disease or condition. One of ordinary skill in the art may empirically determine the effective amount of a particular compound without necessitating undue experimentation. In some embodiments, the term “effective amount” refers to an amount of a therapeutic agent or prophylactic agent to reduce or diminish one or more symptoms of one or more diseases or disorders, such as reducing, preventing, or reversing the learning and/or memory deficits in an individual suffering from Alzheimer's disease etc. In one or more neurological or neurodegenerative diseases, an effective amount of the drug may have the effect of stimulation or induction of neural mitosis leading to the generation of new neurons, i.e., exhibiting a neurogenic effect; prevention or retardation of neural loss, including a decrease in the rate of neural loss, i.e., exhibiting a neuroprotective effect. An effective amount can be administered in one or more administrations.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, long-lasting GLP-1R agonists may inhibit or reduce the activity and/or quantity of activated microglia by about 10%, 20%, 30%, 40%, 50%, 75%, 85%, 90%, 95%, or 99% from the activity and/or quantity of the same cells in equivalent tissues of subjects that did not receive or were not treated with long-lasting GLP-1R agonists. In some embodiments, the inhibition and reduction are compared at mRNAs, proteins, cells, tissues, and organs levels. For example, an inhibition and reduction in the rate of neural loss, in the rate of decrease of brain weight, or in the rate of decrease of hippocampal volume, as compared to an untreated control subject.

The term “treating” or “preventing” refers to amelioration, alleviation or reduction of one or more symptoms of a disease, disorder, or condition in an person who may be predisposed to the disease, disorder and/or condition but has not yet been diagnosed as having it; reducing disease symptoms, inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms of chronic impairment associated with SARS-COV-2 infection are mitigated or eliminated, including reducing the rate of neuronal loss, decreasing symptoms resulting from the disease, increasing the quality of life of those suffering from the disease, decreasing the dose of other medications required to treat the disease, delaying the progression of the disease, and/or prolonging survival of individuals. The term “ameliorate” refers to a decrease, suppression, attenuation, diminish, arrest, or stabilization of the development or progression of a disease.

The term “biodegradable” generally refers to a material that will degrade or erode under physiologic conditions to smaller units or chemical species that are capable of being metabolized, eliminated, or excreted by the subject. The degradation time is a function of composition and morphology.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide, as is described herein.

The term “long-acting GLP-1R agonist” refers to a glucagon like peptide 1 receptor agonists (GLP-1R agonist) which is effective as a GLP-1R agonist for at least one hour, at least six hours, at least twelve hours, at least one day, at least two days, at least one week, at least two weeks, at least one month, or at least two months.

The terms “Coronavirus disease 2019”, “COVID-19”, or “COVID” refer to the disease caused by the human pandemic SARS-COV-2 virus.

The terms “Post-Covid Conditions”, “long-haul COVID”, “long COVID”, “post-COVID syndrome” or “post-acute sequelae of COVID-19 (PASC)” refer to a chronic disease or disorder in a subject resulting from viral infection with the SARS-COV-2 virus, including new or ongoing symptoms that can begin after infection has resolved and may last weeks, months, or even years after initial infection. Long COVID may be defined by symptoms that persist more than six weeks after infection or are newly emergent and persist after infection. The time of initiation or duration of disease may vary.

The term “brain fog” and “cognitive impairment” refer to complications including cognitive dysfunction, fatigue, and memory loss following COVID infection. Methods to identify and assess memory problems, so called “brain fog”, are known in the art, for example, as described in “Cognitive deficits in people who have recovered from COVID-19”, Hampshire A, et al., EClinicalMedicine (2021) 101044 (DOI://doi.org/10.1016/j.eclinm.2021.101044); www.cdc.gov/coronavirus/2019-ncov/hcp/clinical-care/post-covid-assessment-testing.html; Gulick et al. Practical Neurology, pages 19-21; and Frontera, J., Mainali, S., Fink, E. L. et al., Neurocrit Care 33, 25-34 (2020).

The terms “PEGylation” and “PEGylated” refer to the process and product of both covalent and non-covalent attachment or amalgamation of polyoxyalkylene oxide polymers, preferably polyethylene glycol (PEG) chains, to molecules and macrostructures, such as a drug, therapeutic protein, or particle.

The terms “exendin-4” and “exenatide” refer to the Glucagon-like peptide-1 receptor agonist polypeptide having a 39 amino acid sequence set forth in SEQID NO: 1, and having CAS No. 141758-74-9.

### II. Compositions

Microglia are engaged in signaling with neurons through cytokines and neurotransmitters as well as direct neurite contact. When activated, microglial communication may be impeded, leading directly to defects in memory function and neural editing. Neuroinflammation mediated by microglia plays a key role in neurodegenerative diseases like Parkinson's and Alzheimer's disease and is thought to play a role in other forms of cognitive impairment. It has been discovered that neurologic dysfunction associated with viral infection and, in particular, as a long-term consequence of COVID-19 infection, is driven by activated microglia. Microglia are key mediators of inflammation in the central nervous system and have been identified as targets in neurodegenerative diseases like Parkinson's and Alzheimer's disease. Protein aggregates that are indicative of these diseases were found to stimulate microglial activation, leading to a cascade of events including pro-inflammatory cytokine production, neurotoxic astrocyte formation, and ultimately leads to decline in cognitive processes such as memory or motor coordination, depending on the location of neurodegeneration.

Long-acting glucagon like peptide 1 receptor agonists (long-acting GLP-1R agonists), such as exenatide and PEGylated exenatide, reduce and inhibit pathological processes such as microglial activation. Microglial activation as a result of COVID infection is a target for treatment of neurologic complications secondary to viral infection. Other GLP-1R agonists share the same target receptor and should have the same effects on microglia, though their clinical efficacy may not be the same and may differ depending on tissue distribution, blood brain barrier access, receptor kinetics, and exposure at well-tolerated doses.

Considering the roles of microglia and astrocytes in brain inflammatory responses, cognition, and neurodegeneration, they are key targets for therapeutic strategies against latent neurological manifestations observed in some patients with COVID-19, known as “long-haul” COVID.

Long-acting glucagon like peptide 1 receptor agonists reduce and inhibit pathological processes that give rise to chronic disease symptoms following infection with the virus that causes COVID-19. Compositions of long-lasting GLP-1R agonists and pharmaceutical formulations thereof are provided for treating and preventing symptoms of long COVID. In preferred embodiments, the long-lasting GLP-1R agonists are PEGylated GLP-1R agonists. The compositions are administered in an amount for administration effective to alleviate or prevent one or more symptoms of COVID in subjects in need thereof.

### A. Long Acting GLP-1R Agonists

Compositions and pharmaceutical formulations of long-lasting GLP-1R agonists are provided. An exemplary long acting-GLP-1R agonist is a modified short-acting GLP-1R agonist that is modified to enhance plasma half-life. A preferred modified short-acting GLP-1R agonist is PEGylated exenatide. In other embodiments, the long-acting GLP-1 agonist includes an Fc-fusion GLP-1 (e.g., dulaglutide, efpeglenatide) or derivative thereof.

In some embodiments, the long-acting GLP-1 agonist is an albumin-fusion GLP-1 (e.g., albiglutide) or derivative thereof. In some embodiments, the Fc-fusion GLP-1 composition is dulaglutide. Dulaglutide is a glucagon-like peptide 1 receptor agonist (GLP-1 agonist) for the treatment of type 2 diabetes that can be used once weekly. Dulaglutide includes GLP-1 (7-37) covalently linked to an Fc fragment of human IgG4, thereby protecting the GLP-1 moiety from inactivation by dipeptidyl peptidase 4. GLP-1 is a hormone that is involved in the normalization of level of glucose in blood (glycemia). GLP-1 is normally secreted by L cells of the gastrointestinal mucosa in response to a meal. Dulaglutide binds to glucagon-like peptide 1 receptors, slowing gastric emptying and increases insulin secretion by pancreatic Beta cells. Simultaneously the compound reduces the elevated glucagon secretion by inhibiting alpha cells of the pancreas, which is known to be inappropriate in the diabetic patient.

In other embodiments, the long-acting GLP-1 agonist of an albumin-fusion of GLP-1 composition is albiglutide. Albiglutide is a glucagon-like peptide-1 agonist (GLP-1 agonist) drug used for treatment of type 2 diabetes. It is a dipeptidyl peptidase-4-resistant glucagon-like peptide-1 dimer fused to human albumin. Albiglutide has a half-life of four to seven days.

### 1. PEGylated Exenatide

In preferred embodiments, the modified short-acting GLP-1R agonist is PEGylated exenatide.

a. Exenatide

Exenatide (Exendin-4) is a peptide agonist of GLP-1R that facilitates insulin release in type two diabetes (T2D) and is marketed as BYETTA® for T2D (Meier, J J, Nat Rev Endocrinol, 2012. 8 (12): p. 728-42). Also known as “exendin-4” and marketed as “BYETTA®” and “BYDUREON®” exenatide is an engineered Glucagon-like peptide-1 receptor agonist peptide drug having CAS No. 141758-74-9. Exenatide is a 39-amino-acid peptide, an insulin secretagogue, with glucoregulatory effects.

The peptide sequence of exenatide is:

(SEQ ID NO: 1)
HGEGTFTSDLSKQMEEEAVRLFIEWLKNGGPSSGAPPPS.

This peptide drug manages insulin release in a glucose-dependent manner and is therefore safe for non-diabetic patients. Exenatide also reduces a range of neurodegenerative processes (Holscher, C., J Endocrinol, 2014. 221 (1): p. T31-41). In preclinical models, exenatide crosses the blood brain barrier (BBB), protects memory formation in AD or motor activity in PD, protects synapses and synaptic functions, enhances neurogenesis, reduces apoptosis, protects neurons from oxidative stress, as well as reduces plaque formation and the chronic inflammation response in the brains of AD and PD mouse models. Exenatide, like other peptide drugs, is inherently short-lived and unstable in the blood stream and therefore requires frequent injections.

Exenatide belongs to the group of incretin mimetics, approved in April 2005 for the treatment of diabetes mellitus type 2. Exenatide in its BYETTA® form is administered as a subcutaneous injection (under the skin) of the abdomen, thigh, or arm, any time within the 60 minutes before the first and last meal of the day. Exenatide was approved by the FDA on Apr. 28, 2005 for patients whose diabetes was not well-controlled on other oral medication. The medication is injected subcutaneously twice per day using a filled pen-like device. A once-weekly injection has been approved as of Jan. 27, 2012 under the trademark BYDUREON®. It is manufactured by Amylin Pharmaceuticals and commercialized by Astrazeneca.

Exenatide is a synthetic version of Exendin-4, a hormone found in the saliva of the Gila monster. It displays biological properties similar to human glucagon-like peptide-1 (GLP-1), a regulator of glucose metabolism and insulin secretion. According to the package insert, exenatide enhances glucose-dependent insulin secretion by the pancreatic beta-cell, suppresses inappropriately elevated glucagon secretion, and slows gastric emptying, although the mechanism of action is still under study.

The incretin hormones GLP-1 and glucose-dependent insulinotropic peptide (GIP) are produced by the L and K endocrine cells of the intestine following ingestion of food. GLP-1 and GIP stimulate insulin secretion from the beta cells of the islets of Langerhans in the pancreas. Only GLP-1 causes insulin secretion in the diabetic state; however, GLP-1 itself is ineffective as a clinical treatment for diabetes as it has a very short half-life in vivo. Exenatide bears a 50% amino acid homology to GLP-1 and it has a longer half-life in vivo. Thus, it was tested for its ability to stimulate insulin secretion and lower blood glucose in mammals and was found to be effective in the diabetic state. In studies on rodents, it has also been shown to increase the number of beta cells in the pancreas.

Commercially, exenatide is produced by direct chemical synthesis. Historically, exenatide was discovered as Exendin-4, a protein naturally secreted in the saliva and concentrated in the tail of the Gila monster. Exendin-4 shares extensive homology and function with mammalian GLP-1 but has a therapeutic advantage in its resistance to degradation by DPP-IV (which breaks down GLP-1 in mammals) therefore allowing for a longer pharmacological half-life. The biochemical characteristics of Exendin-4 enabled consideration and development of exenatide as a diabetes mellitus treatment strategy. Subsequent clinical testing led to the discovery of the also desirable glucagon and appetite-suppressant effects.

In its twice daily BYETTA® form, exenatide raises insulin levels quickly (within about ten minutes of administration) with the insulin levels subsiding substantially over the next hour or two. A dose taken after meals has a much smaller effect on blood sugar than one taken beforehand. The effects on blood sugar diminish after six to eight hours. In its BYETTA® form, the medicine is available in two doses: 5 mcg. and 10 mcg. Treatment often begins with the 5 mcg. dosage, which is increased if adverse effects are not significant. Its once weekly BYDUREON® form is unaffected by the time between the injection and when meals are taken. BYDUREON® has the advantage of providing 24-hour coverage for blood sugar lowering, while BYETTA® has the advantage of providing better control of the blood sugar spike that occurs right after eating. Per the FDA label for BYDUREON®, BYDUREON® lowers HbA1c blood sugar by an average of 1.6%, while BYETTA® lowers it by an average of 0.9%. Both BYETTA® and BYDUREON® have similar weight loss benefits. Per the FDA approved BYDUREON® label, the levels of nausea are lower for BYDUREON® patients than for BYETTA® patients.

b. Polyalkylene Oxide

Polyalkylene oxide, such as polyethylene glycol (PEG), is a polyether compound with many applications from industrial manufacturing to medicine. The structure of PEG is (note the repeated element in parentheses): H—(O—CH₂—CH₂) n-OH PEG is also known as polyethylene oxide (PEO) or polyoxyethylene (POE), depending on its molecular weight. PEG, PEO, or POE refers to an oligomer or polymer of ethylene oxide. The three names are chemically synonymous, but historically PEG is preferred in the biomedical field, whereas PEO is more prevalent in the field of polymer chemistry. Because different applications require different polymer chain lengths, PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g., viscosity) due to chain length effects, their chemical properties are nearly identical. Different forms of PEG are also available, depending on the initiator used for the polymerization process. The most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Very high purity PEG has recently been shown to be crystalline, allowing determination of a crystal structure by x-ray diffraction. Since purification and separation of pure oligomers is difficult, the price for this type of quality is often 10-1000 fold that of polydisperse PEG.

PEGs are also available with different geometries. Branched PEGs have three to ten PEG chains emanating from a central core group. Star PEGs have 10 to 100 PEG chains emanating from a central core group. Comb PEGs have multiple PEG chains normally grafted onto a polymer backbone. The numbers that are often included in the names of PEGs indicate their average molecular weights (e.g., a PEG with n=9 would have an average molecular weight of approximately 400 Daltons and would be labeled PEG 400. Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). MW and Mn can be measured by mass spectrometry.

PEGylation is the act of covalently coupling a PEG structure to another larger molecule, for example, a therapeutic protein, which is then referred to as a PEGylated protein. PEGylated interferon alfa-2a or -2b are commonly used as injectable treatments for Hepatitis C infection. PEG is soluble in water, methanol, ethanol, acetonitrile, benzene, and dichloromethane, and is insoluble in diethyl ether and hexane. It is coupled to hydrophobic molecules to produce non-ionic surfactants. PEGs contain potential toxic impurities, such as ethylene oxide and 1,4-dioxane. Ethylene Glycol and its ethers are nephrotoxic if applied to damaged skin.

Polyethylene glycol (PEG) and related polymers (PEG phospholipid constructs) are often sonicated when used in biomedical applications. However, PEG is very sensitive to sonolytic degradation and PEG degradation products can be toxic to mammalian cells. It is, thus, imperative to assess potential PEG degradation to ensure that the final material does not contain undocumented contaminants that can introduce artifacts into experimental results.

PEGs and methoxypolyethylene glycols vary in consistency from liquid to solid, depending on the molecular weight, as indicated by a number following the name. They are used commercially in numerous applications, including as surfactants, in foods, in cosmetics, in pharmaceutics, in biomedicine, as dispersing agents, as solvents, in ointments, in suppository bases, as tablet excipients, and as laxatives. Some specific groups are lauromacrogols, nonoxynols, octoxynols, and poloxamers.

Polyethylene glycol is produced by the interaction of ethylene oxide with water, ethylene glycol, or ethylene glycol oligomers. The reaction is catalyzed by acidic or basic catalysts. Ethylene glycol and its oligomers are preferable as a starting material instead of water, because they allow the creation of polymers with a low polydispersity (narrow molecular weight distribution). Polymer chain length depends on the ratio of reactants.

HOCH₂CH₂OH+n(CH₂CH₂O)→HO(CH₂CH₂O)n+1H

Depending on the catalyst type, the mechanism of polymerization can be cationic or anionic. Polymerization of ethylene oxide is an exothermic process.

Polyethylene oxide, or high-molecular weight polyethylene glycol, is synthesized by suspension polymerization. It is necessary to hold the growing polymer chain in solution in the course of the polycondensation process. The reaction is catalyzed by magnesium-, aluminium-, or calcium-organoelement compounds. To prevent coagulation of polymer chains from solution, chelating additives such as dimethylglyoxime are used. Alkali catalysts such as sodium hydroxide (NaOH), potassium hydroxide (KOH), or sodium carbonate (Na₂CO₃) are used to prepare low-molecular-weight polyethylene glycol.

PEG is used as an excipient in many pharmaceutical products. Lower-molecular-weight variants are used as solvents in oral liquids and soft capsules, whereas solid variants are used as ointment bases, tablet binders, film coatings, and lubricants. PEG is also used in lubricating eye drops.

Polyethylene glycol has a low toxicity and is used in a variety of products. The polymer is used as a lubricating coating for various surfaces in aqueous and non-aqueous environments. Since PEG is a flexible, water-soluble polymer, it can be used to create very high osmotic pressures (on the order of tens of atmospheres). It also is unlikely to have specific interactions with biological chemicals. These properties make PEG one of the most useful molecules for applying osmotic pressure in biochemistry and biomembranes experiments, in particular when using the osmotic stress technique.

PEGylation (also often styled pegylation) is the process of both covalent and non-covalent attachment or amalgamation of polyethylene glycol (PEG) polymer chains to molecules and macrostructures, such as a drug, therapeutic protein, or vesicle, which is then described as PEGylated (pegylated). PEGylation is routinely achieved by incubation of a reactive derivative of PEG with the target molecule. The covalent attachment of PEG to a drug or therapeutic protein can “mask” the agent from the host's immune system (reduced immunogenicity and antigenicity), and increase the hydrodynamic size (size in solution) of the agent which prolongs its circulatory time by reducing renal clearance. PEGylation can also provide water solubility to hydrophobic drugs and proteins.

PEGylation is the process of attaching the strands of the polymer PEG to molecules, most typically peptides, proteins, and antibody fragments, that can improve the safety and efficiency of many therapeutics. It produces alterations in the physiochemical properties including changes in conformation, electrostatic binding, hydrophobicity etc. These physical and chemical changes increase systemic retention of the therapeutic agent. Also, it can influence the binding affinity of the therapeutic moiety to the cell receptors and can alter the absorption and distribution patterns.

PEG is a particularly attractive polymer for conjugation. The specific characteristics of PEG moieties relevant to pharmaceutical applications are: water solubility, high mobility in solution, lack of toxicity and low immunogenicity, ready clearance from the body, and altered distribution in the body.

Although PEGylation is a gold standard method to extend the half-life of protein drugs (Harris, J. M. and R. B. Chess, Nat Rev Drug Discov, 2003. 2 (3): p. 214-21), it is generally not applied to smaller peptide drugs, because conjugation with a large PEG molecule often diminishes the biological activity of the peptide (e.g., to less than 1% biological activity vs. native peptide). A suitable PEGylation technology that extends circulating half-lives of short-acting peptides while simultaneously preserving therapeutic activities of exenatide is described in WO2013002580 and US20130217622. In preferred embodiments, the PEGylated exenatide is NLY01.

### 2. NLY01

A subcutaneously administered, long-acting exenatide has been developed that, despite having a large molecular weight, has high bioactivity in the brain and significant neuroprotective and disease-modifying effects in mouse models of central nervous system disorders, e.g., neurodegenerative diseases. A PEGylated exenatide (NLY01) with significantly improved half-life and mean residence time in non-human primates compared to BYETTA® exenatide (exendin-4 which has a 2-hour half-life) and liraglutide (which has a 13 hour half-life) s described. PEGylated exenatide maintains its biological activity by a site specifically attached polyethylene glycol (PEG) molecule to exenatide (WO2013002580). Due to its greater half-life and potency, this compound is suitable for a once-weekly, bi-monthly or once-monthly clinical dosing frequency. This dosing frequency is an improvement over the current twice daily treatment (exenatide, BYETTA®) or once-daily treatment (liraglutide, VICTOZA®).

NLY01, as a long-acting PEGylated form of exenatide, has an extended half-life of 12±4 days in humans and 88 hours in most primates. The long-acting features of NLY01 are engineered through a half-life extension technology which still allows the composition to follow the same target and mechanism of action as exenatide.

As a long-acting exenatide-based therapy, NLY01 offers an improved drug delivery approach compared to exenatide, while maintaining its pharmacological effects. For example, as described in detail below, similar to exenatide, NLY01 improves motor and cognitive symptoms in PD. Unlike exenatide therapies identified prior to the methods described herein, NLY01 is delivered to patients with a single or bi-monthly injection, preventing daily multiple injections and improving compliance to therapy.

Because NLY01 has a large molecular weight poly(ethylene glycol) polymer (PEG, 50,000 Da) conjugated to the small exenatide peptide (˜4,000 Da), similar pharmacological efficacy to exenatide in Parkinson's Disease (PD) and Alzheimer's Disease (AD) was completely unexpected because of the likely inability to cross the blood-brain barrier (BBB) (Pardridge, W. M., NeuroRx, 2005. 2 (1): p. 3-14). NLY01 traverses the blood-brain barrier (BBB) and modulates the activity of activated microglia.

a. Traversing the Blood Brain Barrier (BBB)

NLY01 traverses the blood brain barrier without the use of targeting or trafficking moieties.

The blood-brain barrier (BBB) is a highly selective permeability barrier that separates the circulating blood from the brain extracellular fluid (BECF) in the central nervous system (CNS). The blood-brain barrier is formed by brain endothelial cells, which are connected by tight junctions with an extremely high electrical resistivity. Astrocytes are necessary to create the blood-brain barrier. The blood-brain barrier allows the passage of lipid-soluble molecules, water, and some gases by passive diffusion, as well as the selective transport of molecules such as amino acids and glucose which are crucial to neural function. The blood-brain barrier occurs along all brain capillaries and includes tight junctions around the capillaries that do not exist in normal circulation. Endothelial cells restrict the diffusion of microscopic objects (e.g., bacteria) and large or hydrophilic molecules into the cerebrospinal fluid (CSF), while allowing the diffusion of small hydrophobic molecules (e.g., O₂, CO₂, hormones). Cells of the barrier actively transport metabolic products such as glucose across the barrier with specific proteins. This “barrier” results from the selectivity of the tight junctions between endothelial cells in CNS vessels that restricts the passage of solutes. At the interface between blood and the brain, endothelial cells are stitched together by these tight junctions, which are composed of smaller subunits, frequently biochemical dimers, that are transmembrane proteins such as occludin, claudins, junctional adhesion molecule (JAM), or ESAM, for example. Each of these transmembrane proteins is anchored into the endothelial cells by another protein complex that includes zo-1 and associated proteins.

The blood-brain barrier is formed by the brain capillary endothelium and excludes from the brain approximately 100% of large-molecule neurotherapeutics and more than 98% of all small-molecule drugs. Overcoming the difficulty of delivering therapeutic agents to specific regions of the brain presents a major challenge to treatment of most brain disorders. In its neuroprotective role, the blood-brain barrier functions to hinder the delivery of many potentially important diagnostic and therapeutic agents to the brain. Therapeutic molecules and antibodies that might otherwise be effective in diagnosis and therapy do not cross the BBB in adequate amounts. Mechanisms for drug targeting in the brain involve going either “through” or “behind” the BBB. Modalities for drug delivery/Dosage form through the BBB entail its disruption by osmotic means; biochemically by the use of vasoactive substances such as bradykinin; or even by localized exposure to high-intensity focused ultrasound (HIFU). Other methods used to get through the BBB may entail the use of endogenous transport systems, including carrier-mediated transporters such as glucose and amino acid carriers; receptor-mediated transcytosis for insulin or transferrin; and the blocking of active efflux transporters such as p-glycoprotein. However, vectors targeting BBB transporters, such as the transferrin receptor, have been found to remain entrapped in brain endothelial cells of capillaries, instead of being ferried across the BBB into the cerebral parenchyma. Methods for drug delivery behind the BBB include intracerebral implantation (e.g., using needles) and convection-enhanced distribution. Additionally, mannitol can be used in bypassing the BBB.

b. Modulation of Activated Microglia

In some embodiments, NLY01 functions to ameliorate neurological diseases and disorders by reducing the activation of microglia. In response to neurodegeneration and the accumulation of abnormally aggregated proteins, such as α-synuclein and β-amyloid, resting microglia become an activated state and release various cytokines and neurotoxic molecules including TNF-α, IL-1α, IL-1β, IL-6, and C1q that drive their proliferation and activate astrocytes (A1 astrocytes). Consequently, such inflammatory mediators released from activated microglia or reactive astrocytes, induced by activated microglia, causes neuronal damage, and contribute to the progression of neurodegenerative diseases. Therefore, activated microglia can be described as major upstream bad actors in neurodegenerative diseases. Inhibition of microglia activation without off-target toxicity is a logical strategy to prevent, stop and/or reverse the neurodegeneration process. However, prior to the methods described herein, the lack of translational methods to specifically target microglia activation hampered this strategy.

The studies describe a unique strategy to selectively target and block microglia and astrocytes activation and the release of inflammatory and neurotoxic molecules from activated resident innate immune cells; thus prevent, stop and/or ameliorate the progression of cognitive conditions associated with long COVID. It was discovered that microglia activated by abnormally aggregated proteins upregulate GLP-1r and a long-acting GLP-1r agonist bound to activated microglia significantly inhibit the release of toxic molecules including TNF-α, IL-1α, IL-1β, IL-6, and C1q and protect neurons. A long-acting GLP-1r agonist demonstrates slow internalization of GLP-1r, reducing the rate of GLP-1r recycling compared to that of short-acting GLP-1r agonists (exenatide and liraglutide), and thus can continuously activate GLP-1r and induce GLP-1r signaling in the brain. Patients treated with a short-acting GLP-1r agonist would experience “off time” that will mar the therapeutic effect during a chronic treatment. In contrast, NLY01 has the ability to penetrate BBB and activate GLP-1r in the brain in a continuous fashion without “off time” and without off-target toxicity. In Tg models of Alzheimer's Disease (AD) NLY01 treatment ameliorated memory impairment and reduced amyloid aggregation and tau formation, the hallmark of AD. Consistent with PD studies, NLY01 demonstrated significantly inhibited microglia activation and the population of reactive astrocytes in the AD brain.

### 3. Other GLP-1R Agonists

In some embodiments, compositions include one or more GLP-1R agonists that is not exenatide, or an analog or derivative thereof. Exemplary GLP-1R agonists include GLP-1R agonists approved for treatment of type 2 diabetes, like Victoza (liraglutide), and Ozempic (semaglutide) can, acting through the GLP-1R on microglia, reduce activation in COVID patients thereby proving therapeutic in the treatment of neurological symptoms which persist or emerge after infection. Therefore, in some embodiments the one or more GLP-1R agonists are liraglutide and semaglutide.

Other examples of therapeutic active agents include DPPIV inhibitors, which increase native GLP-1R levels by inhibiting the protease dipeptidyle peptidase IV, which is responsible for rapid inactivation of GLP-1 in circulation. Medicines in the DPP-4 inhibitor class include Januvia (sitagliptin), Onglyza (saxagliptin), Tradjenta (linagliptin), and Nesina (alogliptin). Each of these is also available as a combination product with other anti-diabetic drugs such as metformin. Enhancement of native GLP-1 levels through inhibition of DPPIV is another means of targeting the GLP-1R to reduce microglial activation resulting from viral infection and causing lasting neurologic complications after COVID infection. Therefore, in some embodiments the GLP-1R agonist is a DPPIV inhibitor, for example, sitagliptin, saxagliptin, linagliptin, or alogliptin.

### B. Additional Active Agents

In some embodiments the long acting GLP-1R agonists are administered to a subject together with one or more additional active agents, particularly one or more antiviral or antimicrobial agents, or additional agents to prevent or treat one or more symptoms of a neurological or neurodegenerative disease or disorder. Suitable therapeutic, diagnostic, and/or prophylactic agents can be a biomolecule, such as peptides, proteins, carbohydrates, nucleotides or oligonucleotides, or a small molecule agent (e.g., molecular weight less than 2000 amu, preferably less than 1500 amu), including organic, inorganic, and organometallic agents.

### 1. Therapeutic and Prophylactic Agents

In some embodiments, the long acting GLP-1R agonists are administered to a subject together with one or more additional therapeutic, prophylactic, or prognostic agents. Representative therapeutic agents include, but are not limited to, neuroprotective agents, anti-inflammatory agents, antioxidants, anti-infectious agents, and combinations thereof.

In one embodiment, the additional agent is a steroid. Suitable steroids include biologically active forms of vitamin D3 and D2, such as those described in U.S. Pat. Nos. 4,897,388 and 5,939,407. The steroids may be co-administered to further aid in neurogenic stimulation or induction and/or prevention of neural loss. Estrogen and estrogen related molecules such as allopregnanolone can be co-administered with the neuro-enhancing agents to enhance neuroprotection, as described in Brinton (2001) Learning and Memory 8 (3): 121-133.

Other neuroactive steroids, such as various forms of dehydroepi-androsterone (DHEA), as described in U.S. Pat. No. 6,552,010, can also be co-administered to further aid in neurogenic stimulation or induction and/or prevention of neural loss. Other agents that cause neural growth and outgrowth of neural networks, such as Nerve Growth Factor (NGF) and Brain-derived Neurotrophic Factor (BDNF), can be administered either simultaneously with or before or after the administration of THP. Additionally, inhibitors of neural apoptosis, such as inhibitors of calpains and caspases and other cell death mechanisms, such as necrosis, can be co-administered with the neuro-enhancing agents to further prevent neural loss associated with certain neurological diseases and neurological defects.

### C. Pharmaceutical Excipients

In some embodiments, the long-acting GLP-1R agonists are formulated with one or more pharmaceutical excipients, additives, or fillers. For example, in some embodiments, the long-acting GLP-1R agonists are formulated into pharmaceutical formulations for administration to a subject. Compositions including long-acting GLP-1R agonists may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain long-acting GLP-1R agonists in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### 1. Dosage Units

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### 2. Formulations for Administration

In certain embodiments, the compositions of long-acting GLP-1R agonists are formulated into a pharmaceutically acceptable formulation for administration via a specific route. In some embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in some embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

a. Parenteral Administration

In certain embodiments, long-acting GLP-1R agonists are formulated into a pharmaceutically acceptable formulation for parenteral administration. The phrases “parenteral administration” and “administered parenterally” are art-recognized terms, and include modes of administration other than enteral and topical administration, such as injections, and include without limitation intravenous (i.v.), intramuscular (i.m.), intraperitoneal (i.p.), subcutaneous (s.c.) injection and infusion. The long-acting GLP-1R agonists can be administered parenterally, for example, by intravenous, intraperitoneal, or subcutaneous routes.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The long-acting GLP-1R agonists can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

b. Enteral Administration

In some embodiments, long-acting GLP-1R agonists are administered orally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

### III. Methods of Making Long-Acting GLP-1R Agonists

### A. Methods of Making NLY01

Long-acting GLP-1R agonists can be prepared via a variety of chemical reaction steps. Typically, methods for making Long-acting GLP-1R agonists includer PEGylation.

### 1. PEGylation

The first step of the PEGylation is the functionalization of the PEG polymer at one or both terminals. PEGs that are activated at each terminus with the same reactive moiety are known as “homobifunctional”, whereas if the functional groups present are different, then the PEG derivative is referred as “heterobifunctional” or “heterofunctional.” The chemically active or activated derivatives of the PEG polymer are prepared to attach the PEG to the desired molecule.

The overall PEGylation processes for protein conjugation can be broadly classified into two types, namely a solution phase batch process and an on-column fed-batch process. The simple and commonly adopted batch process involves the mixing of reagents together in a suitable buffer solution, preferably at a temperature between 4° and 6° C., followed by the separation and purification of the desired product using a suitable technique based on its physicochemical properties, including size exclusion chromatography (SEC), ion exchange chromatography (IEX), hydrophobic interaction chromatography (HIC) and membranes or aqueous two-phase systems.

The choice of the suitable functional group for the PEG derivative is based on the type of available reactive group on the molecule that will be coupled to the PEG. For proteins, typical reactive amino acids include lysine, cysteine, histidine, arginine, aspartic acid, glutamic acid, serine, threonine, tyrosine. The N-terminal amino group and the C-terminal carboxylic acid can also be used as a site-specific site by conjugation with aldehyde functional polymers. The techniques used to form first generation PEG derivatives are generally reacting the PEG polymer with a group that is reactive with hydroxyl groups, typically anhydrides, acid chlorides, chloroformates and carbonates. In the second generation PEGylation chemistry more efficient functional groups such as aldehyde, esters, amides etc. made available for conjugation.

Heterobifunctional PEGs are useful in linking two entities, where a hydrophilic, flexible and biocompatible spacer is needed. Preferred end groups for heterobifunctional PEGs are maleimide, vinyl sulfones, pyridyl disulfide, amine, carboxylic acids and NHS esters. Third generation pegylation agents, where the shape of the polymer has been branched, Y shaped or comb shaped are available which show reduced viscosity and lack of organ accumulation. Unpredictability in clearance times for PEGylated compounds may lead to the accumulation of large molecular weight compounds in the liver leading to inclusion bodies with no known toxicologic consequences. Furthermore, alteration in the chain length may lead to unexpected clearance times in vivo.

a. PEGylation of GLP-1r Agonist (NLY01, PB-119, LY2428757)

The half-life of an Exendin-4 analogue (e.g., exenatide) or GLP-1 analogue can be increased via the selective PEGylation, allowing less frequent and/or lower doses to be administered. Pegylation increases molecular weight, defense of a metabolism site and inhibition of an immunogenicity site, increasing in vivo half-life and stability and reducing immunogenicity. Furthermore, kidney excretion of peptides and proteins bound with PEG is reduced due to the increase of molecular weights of peptides and proteins by PEG, so that PEGylation has advantages of increasing effects in both pharmacokinetically and pharmacodynamically.

PEGs can be a linear type or a branched type, preferably a dimeric type or a trimeric polymer, most preferably a trimeric polymer. Exemplary polyethylene glycol derivatives include methoxypolyethylene glycol succinimidylpropionate, methoxypolyethylene glycol N-hydroxysuccinimide, methoxypolyethylene glycol propionaldehyde, methoxypolyethylene glycol maleimide, and multiple branched types thereof. Preferably, the polyethylene glycol derivative is linear methoxypolyethylene glycol maleimide, branched methoxypolyethylene glycol maleimide or trimeric methoxypolyethylene glycol maleimide, most preferably is trimeric methoxypolyethylene glycol maleimide.

After the Exendin-4 analogue (e.g., exenatide) is PEGylated with polyethylene glycol or the derivative thereof is prepared, the molecular structure of the analogue can be confirmed by a mass spectroscope, a liquid chromatography, an X-ray diffraction analysis, a polarimetry, and comparison between calculated values and measured values of representative elements constituting the PEGylated exenatide.

When the composition is used as medication, the pharmaceutical composition containing the Exendin-4 analogue (e.g., exenatide) PEGylated with polyethylene glycol or a derivative thereof is formulated into various oral or non-oral administration forms as the following in case of clinical administration, but is not limited thereof.

The pharmaceutical composition containing the Exendin-4 analogue PEGylated with polyethylene glycol or a derivative thereof may be parenterally administrated, for example, by subcutaneous injection, intravenous injection, intramuscular injection, intrathoracic injection, or topical administration.

The Exendin-4 analogue (e.g., exenatide) PEGylated with polyethylene glycol or a derivative thereof may be prepared as a liquid or suspension by mixing it with stabilizer or buffer in water to formulize it into parenterally administration purposed formulation, and this may be prepared into ampoule or vial unit administration form. The composition is sterilized and/or may include adjuvants such as antiseptics, stabilizers, hydrators or emulsify stimulators, osmotic pressure controlling purposed salts and/or buffers, and other substances beneficial for treatments, formulated according to traditional methods of mixture, granulation or coating.

The effective dose of the pharmaceutical composition containing the Exendin-4 analogue (e.g., exenatide) PEGylated with polyethylene glycol or a derivative thereof will vary depending on the age, body weight, gender, administration form, health status and level of disease of patients, and may be administrated via oral or non-oral route following decisions of doctors or pharmacists, preferably at a dose of 0.01 to 200 mg/kg/day.

### IV. Methods of Use

Methods of using the long-acting glucagon like peptide 1 receptor agonists (GLP-1R agonists) are described. Typically, the long-acting GLP-1R agonists are PEGylated GLP-1R agonists (P-GLP-1R agonists).

It has been established that P-GLP-1R agonists target activated microglia in the brain to reduce or prevent neurological diseases or disorders in a subject. Methods of using P-GLP-1R agonists for treating or preventing chronic neurological diseases or disorders resulting from prior infection with a SARS virus are useful to treat and/or prevent cognitive impairment associated with viral infection, for example, infection with SARS-COV-2 virus. In preferred embodiments, cognitive impairment associated with COVID in a subject is treated by administering an effective amount of NLY01 to the subject. The NLY01 is administered in an amount and with a dosing regimen effective to prevent, inhibit, or reduce one or more cognitive symptoms associated with COVID in the subject. The NLY01 is administered to a subject in one or multiple doses, at one or multiple time points following an initial dose. The effects of the methods can be compared to a reference or control.

Treatment of any disease where microglial activation leads to memory or other neurologic dysfunction can be envisioned as a target for NLY01 treatment. For example, fibromyalgia is a disorder of chronic generalized muscular pain, stiffness, generalized fatigue, sleep abnormalities, (Clauw D J et al., Mayo Clin Proc. 2011 September; 86 (9): 907-11; Schmidt-Wilcke and Clauw, Nat Rev Rheumatol. 2011 Jul. 19; 7 (9): 518-27) and cognitive problems (Theoharides T C et al., J Pharmacol Exp Ther. 2015 November; 355 (2): 255-63). While the etiology of fibromyalgia is not entirely clear, it appears chronic microglial activation may be pathologic. A recent study found that clinical fatigue scores in these patients correlated with increased glial activation as assessed by TSPO-PET imaging (Albrecht D S et al., Brain Behav Immun. 2019; 75:72-83). In addition to its promise in the treatment of post-COVID neurologic complications, NLY01 may, by reducing microglial activation, also prove an effective treatment for fibromyalgia.

A. Methods of Treating Cognitive Deficits Associated with Viral Infections

Methods for treating, alleviating, and/or preventing one or more pathological processes and/or symptoms of neurological impairment associated with viral infection in a subject are provided. The methods include systemically administering to the subject an effective amount of a PEGylated exenatide, or a PEGylated exenatide analog, to treat, alleviate, and/or prevent one or more pathological processes and/or symptoms of neurological impairment associated with viral disease. Preferably, the compositions decrease secretion of pro-inflammatory cytokines by activated microglia in the brain, improve cognition, or combinations thereof; inhibit or reduce activity and/or quantity of activated microglia; or reduce symptoms of neurological impairment associated with viral disease. The methods can include identifying a subject having one or more biological markers associated with development of neurological impairment associated with viral disease.

In some embodiments, the methods are used to effectively reduce cognitive or neurological impairment associated with viral infection in a subject in need thereof. In some embodiments, the methods reverse neurological defects and/or cognitive impairment associated with microglial activation elicited by prior infection with a virus, such that neurological function and/or cognitive ability in the subject are equivalent to that prior to the infection, or to the initiation of the defect and/or impairment. In some embodiments, the amount of P-GLP-1R agonists administered to the subject is selected to deliver an effective amount to reduce, prevent, or otherwise alleviate one or more clinical or molecular symptoms of neurological disease or disorder associated with viral infection, as compared to a control.

An exemplary viral infection associated with neurological symptoms is infection with SARS-COV-2. For example, in some embodiments, the subject has Long-COVID. Other viruses that cause neurological symptoms due to activation of microglia include those of the Flaviviridae family, the Togaviridae family, and Rhabdoviridae family.

### 1. COVID-19 and Long COVID

Methods for treating, alleviating, and/or preventing one or more pathological processes and/or symptoms of cognitive impairment associated with COVID in a subject are provided. The methods include systemically administering to the subject an effective amount of a PEGylated exenatide, or a PEGylated exenatide analog, to treat, alleviate, and/or prevent one or more pathological processes and/or symptoms associated with long COVID. Preferably, the compositions decrease secretion of pro-inflammatory cytokines by activated microglia in the brain, improve cognition, or combinations thereof; inhibit or reduce activity and/or quantity of activated microglia; or reduce neurological symptoms of long COVID. The methods can include identifying a subject having one or more biological markers associated with development of long COVID.

The PEGylated GLP-1R agonists (P-GLP-1R agonists) and/or pharmaceutical formulations thereof are also suitable for prophylactic use, for example, for preventing one or more symptoms of COVID in a subject who has been infected, or is at risk of being infected with SARS-COV-2. Therefore, in some embodiments, the subject has not experienced cognitive impairment from COVID but is at risk of doing so.

In some embodiments, the methods are used to effectively reduce cognitive or neurological impairment associated with COVID in a subject in need thereof. In some embodiments, the methods reverse neurological defects and/or cognitive impairment associated with microglial activation elicited by prior infection with a SARS-COV-2 virus, such that neurological function and/or cognitive ability in the subject are equivalent to that prior to the infection, or to the initiation of the defect and/or impairment.

In some embodiments, the amount of P-GLP-1R agonists administered to the subject is selected to deliver an effective amount to reduce, prevent, or otherwise alleviate one or more clinical or molecular symptoms of neurological disease or disorder associated with COVID, as compared to a control.

Typically, the subject to be treated is a human. An exemplary subject is one who at increased risk of, or has been infected with a SARS virus, such as the SARS-COV-2 virus, or one who is at risk of, or is suffering from chronic cognitive impairment associated with COVID. All the methods can include the step of identifying and selecting a subject in need of treatment, or a subject who would benefit from administration with the described compositions.

a. Long COVID and Symptoms to be Treated

Chronic impairment associated with SARS virus infection is also known as Post-Covid Conditions, or ‘long-haul COVID’, or ‘long COVID’, or ‘post-COVID syndrome’ or ‘post-acute sequelae of COVID-19 (PASC)’.

A positive SARS-COV-2 viral test (i.e., reverse transcription polymerase chain reaction [RT-PCR] test or antigen test) or serologic (antibody) test can help assess for current or previous infection; however, these laboratory tests are not required to establish a diagnosis of post-COVID conditions. For information about antibody testing, see “Using Antibody Tests for COVID-19.” Healthcare professionals should also consider the possibility of SARS-COV-2 reinfection, especially in persons with new or worsening post-COVID conditions, see “Guidance for SARS-CoV-2 Reinfection.”

Laboratory testing should be guided by the patient history, physical examination, and clinical findings. A basic panel of laboratory tests may be considered for patients with ongoing symptoms (including testing for non-COVID conditions that may be contributing to illness) to assess for conditions that may respond to treatment, until more information and evidence is available for specific laboratory testing for post-COVID conditions. More specialized testing may not be needed in patients who are being initially evaluated for post-COVID conditions; however, expanded testing should be considered if symptoms persist for 12 weeks or longer. The absence of laboratory-confirmed abnormalities or the decision to forgo extensive laboratory testing should not lead to dismissing the possible impact of a patient's symptoms on their daily function. Where clinically indicated, symptom management and a comprehensive rehabilitation plan can be initiated simultaneously with laboratory testing for most patients.

Exemplary tests include monitoring blood count, electrolytes, and renal function, complete blood count with possible iron studies to follow, basic metabolic panel, urinalysis; monitoring liver function, liver function tests or complete metabolic panel; monitoring inflammatory markers, C-reactive protein, erythrocyte sedimentation rate, ferritin; monitoring thyroid function TSH and free T4, and monitoring vitamin deficiencies including vitamin D, vitamin B12.

Millions of people in the US and elsewhere have developed symptoms that persist long after the initial symptoms of COVID-19 resolve. Symptoms can persist for months and include neurologic, respiratory, and other indications. Therefore, in some embodiments, the methods treat one or more symptoms of COVID in a subject who was infected with a virus that causes COVID-19 one, two, three, four, five, six, seven, eight, none, ten or more than ten days, weeks, or months prior to the onset of the one or more symptoms of COVID, typically which are a result of microglial activation caused by the viral infection. In some embodiments, the methods reduce or prevent neurological symptoms of COVID in a subject that has previously been infected with a SARS-COV-2 virus, and has one or more neurological symptoms of COVID, but has no other symptoms associated with COVID-19.

In preferred embodiments, the methods reduce, prevent, or reverse one or more neurological symptoms associated with COVID, following infection with a SARS-COV-2 virus.

SARS-COV-2 infection activates brain microglia and is implicated as a cause of long-term (post-infection) neurologic complications including cognitive dysfunction (aka “brain fog”), fatigue, and memory loss. As a result of memory problems, so called “brain fog”, and other issues, as many as 60% of patients are unable to return to work due to COVID. Symptoms of chronic impairment following SARS virus (Post-Covid Conditions, or ‘long-haul COVID’, or ‘long COVID’, or ‘post-COVID syndrome’ or ‘post-acute sequelae of COVID-19 (PASC)) have been reported to last several months and are estimated to occur in 10 to 30% of COVID-19 patients.

The most common post-covid conditions include fatigue, cognitive difficulties (sometimes called “brain fog”), headache, numbness/tingling, loss of smell or taste, dizziness, heart palpitations, chest pain, shortness of breath, cough, sore throat, joint or muscle pain, excessive sweating, insomnia, depression, anxiety and fever (Nalbandian, A et al., Nat Med 27, 601-615 (2021); A FAIR Health White Paper, Jun. 15, 2021). Therefore, in some embodiments, the methods reduce, prevent, or reverse one or more of fatigue, cognitive difficulties (sometimes called “brain fog”), headache, numbness/tingling, loss of smell or taste, dizziness, heart palpitations, chest pain, shortness of breath, cough, sore throat, joint or muscle pain, excessive sweating, insomnia, depression, anxiety and fever in a subject in need thereof.

Case studies have provided evidence that COVID-19 patients can develop a range of neurological complications including those arising from stroke, encephalopathies, inflammatory syndrome, microbleeds and autoimmune responses. There are concerns regarding potential neurological consequences due to sepsis, hypoxia and immune hyperstimulation, with reports of elevated cerebrospinal fluid autoantibodies in patients with neurological symptoms, white matter change in the brain, and psychological and psychiatric consequences. Therefore, in some embodiments, the methods treat, reduce, or prevent one or more neurological symptoms associated with long COVID in a subject in need thereof.

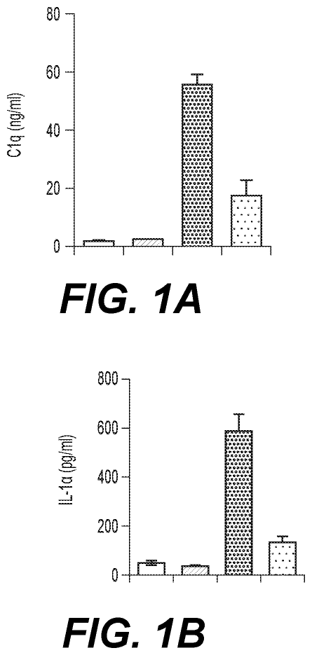

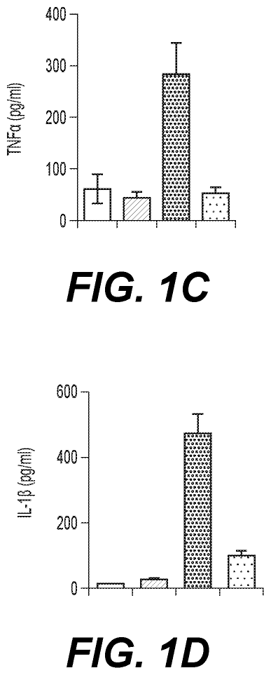

The level of coronavirus neurovirulence in mouse brains and spinal cords has been reported to correlate with its differential ability to induce proinflammatory cytokines in astrocytes and microglia (Li Y et al., J Virol. 2004 April; 78 (7): 3398-406). A recent study of COVID-19 and neurologic complications concluded that significant mechanistic overlap exists between Alzheimer's disease and neurologic complications of COVID-19, centered on neuroinflammation and microvascular injury (see FIG. 1). Therefore, in some embodiments, the methods reduce or prevent the ability to induce proinflammatory cytokines in astrocytes and microglia. In other embodiments, the methods reduce or prevent neuroinflammation and microvascular associated with Long COVID in a subject in need thereof.

i. Neurological Impairment

In some embodiments, the methods reduce, prevent, or reverse one or more symptoms of neurological impairment associated with COVID. COVID-19 infection is associated with cognitive deficits that persist into the recovery phase. Data indicate that long-term neurological manifestations of COVID-19 are relatively frequent, especially in patients who were hospitalized with severe disease, raising questions about mechanism, chronic consequences, and management.

Neurological problems are reported to be the most persistent and common long-term symptoms in COVID patients. Early reports from China suggest neurological symptoms may occur in approximately 36% of COVID-19 positive patients, with increased prevalence among more severe cases. Fatigue was described to be the single most reported symptom and 85% of patients experienced four or more neurologic symptoms, with the most frequent being neurological symptoms (Graham E L et al., Ann Clin Transl Neurol. 2021 May; 8 (5): 1073-1085).

In some embodiments, the methods reduce or prevent neurological symptoms in a subject that has previously been infected with a SARS-COV-2 virus, and has neurological symptoms associated with COVID. In some embodiments, the subject has no other symptoms associated with COVID-19. Typically, the methods treat neurological symptoms in a subject that has previously been infected with a SARS-COV-2 virus, and has neurological symptoms associated with COVID, but has not been diagnosed with a neurodegenerative disease or disorder other than COVID.

Individuals with severe COVID-19 disease can have symptoms of neurological impairment including low energy and cognitive difficulties, such as problems concentrating, disorientation and difficulty finding the right words. Therefore, in some embodiments, the methods treat or prevent one or more symptoms of neurological impairment including low energy, fatigue, and cognitive difficulties, such as problems concentrating, disorientation and difficulty finding the right words, confusion, inattention, and memory loss in a subject in need thereof.

The term “cognitive difficulties” in a subject refers to a reduction in one or more cognitive functions such as reasoning, problem solving, spatial planning and target detection, relative to the cognitive function in the subject prior to the onset of difficulties. In some embodiments, the methods treat, reduce, or prevent cognitive difficulties associated with Long COVID in a subject in need thereof. Therefore, in some embodiments, the methods enhance, increase, or ameliorate one or more cognitive functions such as reasoning, problem solving, spatial planning and target detection in a subject in need thereof.

### Assessing Cognitive Impairment

Neurological impairment can vary in scale with respiratory symptom severity, related to positive biological verification of having had the virus even amongst milder cases. Neurological symptoms of COVID are not explained by differences in age, education or other demographic and socioeconomic variables, and remained in those who had no other residual symptoms and was of greater scale than common pre-existing conditions that are associated with virus susceptibility and cognitive problems.

Methods for testing and identifying cognitive ability in a subject are known in the art, for example, as described in “Cognitive deficits in people who have recovered from COVID-19”, Hampshire A, et al., EClinicalMedicine 000 (2021) 101044 (DOI://doi.org/10.1016/j.eclinm.2021.101044.

Other tests that are used to assess post-COVID cognitive deficits include Neuropsychologic tests such as the Trail Making Test (TMT), Sign Coding Test (SCT), Continuous Performance Test (CPT), and Digital Span Test (DST), as described in Gulick, et al., Practical Neurology: Special report, May 2021 (available online at www.practicalneurology.com/articles/2021-may/special-report-cognitive-screening-after-covid-19).

Other methods for assessing cognitive difficulties include the Montreal Cognitive Assessment (MoCA), Mini Mental Status Examination (MMSE), Compass 31 (for dysautonomia) and Neurobehavioral Symptom Inventory. Additional tests that assess psychological and mental health include General Anxiety Disorder-7 (GAD-7), Patient Health Questionnaire-9 (PHQ-9), PTSD Symptom Scale (PSS), Screen for Posttraumatic Stress Symptoms (SPTSS), PTSD Checklist for DSM-5 (PCL-5), Impact of Event Scale-Revised (IESR), Hospital Anxiety and Depression Scale (HADS) Wood Mental Fatigue Inventory (WMFI), Fatigue Severity Scale, Insomnia Severity Index (ISI), and Connective Tissue Disease Screening Questionnaire (see Evaluating and Caring for Patients with Post-COVID Conditions: Interim Guidance by CDC, available online at www.cdc.gov/coronavirus/2019-ncov/hcp/clinical-care/post-covid-assessment-testing.html).

ii. Microglial Activity

In some embodiments, the methods reduce, prevent, or reverse post-covid neuropathology associated with microglia. Microglia reactivity constitutes an important factor for interindividual differences in response to COVID-19 and for the severity of symptoms observed in COVID-19 patients, especially in older patients or those with comorbidity (Bouayed J et al., J Med Virol. 2021 July; 93 (7): 4111-4113). Increased circulating cytokines during COVID-19 is anticipated to induce or exacerbate microglial reactivity, likely worsening the direct viral or hypoxic injuries in the patients. Chronically reactive microglial state resulting from prolonged systemic cytokine storm might adversely impact the survival of neurons and maintenance of synaptic connections (Andrade E G de et al., Front Cell Neurosci. 2021 Feb. 18; 15:647378). In critical groups such as aged individuals with pre-existing conditions, modulating the pro-inflammatory activity of microglia may help avoid unwanted neurological outcomes. The same holds for individuals with a history of mental health disorders, for which the reduction of dysfunctional microglial synaptic remodeling may prevent the worsening of mental distress associated with the pandemic.

Strong evidence of microglial activation was found in autopsied COVID-19 patients. In a neuropathological, and molecular study of 41 patients with COVID-19 infections, microglial activation with microglial nodules accompanied by neuronophagia (mainly in the brainstem) was observed in the majority (81%) of subjects. No viral RNA or proteins were detected in the brain, suggesting that the observed changes do not result from direct viral infection, but rather from systemic inflammation (Thakur K T et al., Brain. 2021 Apr. 15; awab148). In another post-mortem study in 25 COVID-19 patients, 80% subjects had modifications markedly in the microglial immune activation; 48% exhibited a moderate-to-severe degree of microgliosis and microglial nodules. Significant axonal damage was observed in the cohort, which was more pronounced in patients with microglial nodules. In addition, several microglial clusters displaying high immune activation and expression of microglial functional markers were identified in patients who succumbed to COVID-19 (Schwabenland M et al., Immunity. 2021 Jul. 13; 54 (7): 1594-1610.e11). In a U.S. study from nineteen COVID-19 autopsy cases, 13 exhibited perivascular-activated microglia; in 5 patients activated microglia were detected adjacent to neurons, which is suggestive for neuronophagia in several brain areas (Lee M-H et al., N Engl J Med. 2021 Feb. 4; 384 (5): 481-483). In a post-mortem study conducted in Germany, an increased phagocytic activity was inferred from the microglia phenotype (higher expression of the lysosomal marker CD68). In this study, it was hypothesized that anosmia, a common neurological symptom in COVID-19 patients, was related to pronounced astrogliosis and microgliosis in the olfactory bulb (Matschke J et al., Lancet Neurol 2020 Nov. 1; 19:919).

Taken together, microglia could play the role of a double-edged sword during COVID-19 infections. Microglial reactivity, if initiated effectively, could orchestrate the clearance of SARS-COV-2 in the CNS or trigger neuroinflammation and contribute to the severity of the sequelae associated with viral neurotropism (Thakur K T et al., Brain. 2021 Apr. 15: awab148). Given that the adverse effect of microglial reactivity in the CNS outlasts the direct damaging effect of viral neurotropism, potential incidence of neuropsychiatric disorders and neurodegenerative diseases could be long-lasting in some individuals (Awogbindin I O et al., Front Cell Neurosci. 2021 Jun. 15; 15:670298). The neuroprotective benefits of tetracyclines such as minocycline and doxycycline, have been linked to their microglia suppressant effects (A J M Chaves Filho et al., J Neuroimmune Pharmacol. 2021 June). Furthermore, it has been reported that minocycline attenuates microglia activation and arrests neuro-inflammation resulting in restoration of normal neuronal-microglia communication, controlling the pro-inflammatory profile especially in moderate and severe COVID-19 patients (Oliveira A C et al., Front Neurosci. 2020 Sep. 30; 14:577780). Based on the overall extent of COVID-19 infections and reported incidences of long-term neurological effects, there is an urgent unmet medical need for effective therapies to manage and/or mitigate the COVID-19 CNS effects.

b. Exemplary Treatment Methods

An exemplary method of treatment for chronic symptoms of COVID-19 in subjects in need thereof includes a clinical trial, for example, with a P-GLP-1R agonist (NLY01). POC data should show a reduction in viral-induced microglial activation. An exemplary trial includes a 12-week trial with COG assessments at 0, 6, and 12 weeks vs placebo.

An exemplary trial schedule includes:
Estimated 1-2% patients have persistent cognitive dysfunction post-COVID
Microglial activation found in autopsied COVID-19 patients.
Microglial activation considered potential mediator of long-haul neurologic problems

Some evidence that drugs which reduce microglial activation (minocycline for example) may be therapeutic; however, no formal studies have been conducted. Considering the neuroprotective effects of NLY001, especially through decreased activation of microglia and reduced production of pro-inflammatory cytokines in the brain, NLY001 treatment reduces or even alleviates symptoms of cognitive dysfunction observed in COVID-19 ‘long-haulers’.

### Exemplary Clinical Study Design:

Inclusion: Subjects who recovered from COVID-19 infection (˜2 month post complete recovery from COVID-19 infection) showing cognitive impairment per Montreal Cognitive Assessment (MoCA), MMSE15 or per recommended screening for cognitive symptoms after COVID-19 16
Total subjects: ˜50/group
Treatment groups: NLY01 at 5 mg and Placebo (cross-over study)
Treatment duration: Once weekly for 6 weeks and/or 12 weeks
Observation period: 6 weeks.

### 2. Other Viral Infections

Methods for treating, alleviating, and/or preventing one or more pathological processes and/or symptoms of cognitive impairment associated with viral infections in a subject are provided. The methods include systemically administering to the subject an effective amount of a PEGylated exenatide, or a PEGylated exenatide analog, to treat, alleviate, and/or prevent one or more pathological processes and/or symptoms associated with the viral infection.

Numerous viruses cause brain inflammation leading to neurologic complications. Clinically relevant viruses that cause encephalitis in humans include those of the Flaviviridae family, the Togaviridae family, and Rhabdoviridae family. Rabies virus, which belongs to the Rhabdoviridae family, is a classic example of an acute zoonotic infection that causes 60,000 deaths worldwide every year. Japanese encephalitis virus (JEV), Dengue virus (DenV), West Nile virus (WNV), and Zika virus (ZIKV) belong to the Flaviviridae family.

a. Japanese Encephalitis Virus (JEV)

In some embodiments, the methods reduce, prevent, or reverse neuropathology associated with Japanese encephalitis virus (JEV). JEV is currently restricted to Southeast Asia, where it causes as many as 50,000 infections with a fatality rate as high as 30%. Among survivors of JEV encephalitis infection, 30-50% have significant neurological, cognitive, or psychiatric sequelae.

b. Dengue Virus (DenV)

In some embodiments, the methods reduce, prevent, or reverse neuropathology associated with Dengue virus (DenV). DenV has global prevalence. Estimates suggest that ˜390 million DenV infections occur annually, and in some cases, as many as 21% of these patients present with neurological involvement.

c. West Nile Virus (WNV)

In some embodiments, the methods reduce, prevent, or reverse neuropathology associated with West Nile virus (WNV). WNV is responsible for encephalitis-associated morbidity, mortality, and post-recovery neurocognitive deficits in America. In other embodiments, the methods reduce, prevent, or reverse neuropathology associated with Zika virus (ZIKV). ZIKV may cause similar or different long-term sequelae in survivors. Alphaviruses are enveloped, single-stranded positive-sense RNA viruses of the Togaviridae family that are transmitted by infected biting mosquitos, making them arthropod-borne viruses.

In a study providing prospectively acquired neurological outcomes, the data among American patients with WNV-induced CNS disease showed that during initial clinical presentation, 93% of the patients exhibited a significant neurological deficit, and almost one-half of them had cognitive deficits after a 90-day follow-up. After recovery from the disease, the patients who exhibited neurological deficits 90 days after their initial evaluation constituted only 20% of the study cohort. In a long-term observational study of neurological abnormalities 1-3 and 8-11 years following WNV infection, 86% of patients with WNV encephalitis had abnormal neurological findings at the time of the first assessment, but not uncomplicated fever or meningitis. At the time of the second assessment, approximately 40% of all patients evaluated had developed new neurological complications. This study showed that new neurological complications can develop long after viral clearance. The most common physical, cognitive, and functional sequelae associated with WNV encephalitis are muscle weakness, memory loss, and difficulties in daily living. Other problems include hearing loss and abnormal reflexes. Long-term neurological abnormalities occur most commonly in patients who suffer from WNV encephalitis. Thus, a clinical presentation of West Nile encephalitis (WNE) is associated with serious neurological complications.

d. Viral Encephalitis

In some embodiments, the methods reduce, prevent, or reverse neuropathology associated with Venezuelan, Western, and Eastern equine encephalitis viruses (VEEV, WEEV, and EEEV, respectively). VEEV, WEEV, and EEEV, are major encephalitic alphaviruses that involve high morbidity and neurological sequelae.

CNS inflammation or altered functioning of CNS cells leads to a wide range of behavioral changes. Neurocognitive complications of viral encephalitis include memory disorders, cognitive deficits such as learning disabilities, motor deficits, and changes in mood and personality. A total of 30-50% of people recovering from viral encephalitis develop one or more of these symptoms. Another common result of viral encephalitis is the development of seizures. All of these factors lead to difficulties in daily life.

In some embodiments, the methods treat or prevent one or more symptoms of a neuroviral pathogen associated with neurocognitive impairment. In some embodiments, the methods treat or prevent one or more neurological symptoms of DENV, JEV, Nipah virus, or alphaviruses in a subject in need thereof. Neurological symptoms associated with alphavirus encephalitis include confusion, visual disturbances, photophobia, seizures, somnolence, coma, intellectual disability, and emotional instability/behavioral changes.

### B. Dosage and Effective Amounts

Dosage and dosing regimens are dependent on the severity of the disorder and/or methods of administration, and can be determined by those skilled in the art. A therapeutically effective amount of P-GLP-1R agonist, or pharmaceutical formulation thereof used in the treatment of neurological impairment is typically sufficient to reduce or alleviate one or more symptoms of the neurological impairment.

Preferably, the agents do not target or otherwise modulate the activity or quantity of healthy cells not within or associated with the diseased or target tissues, or do so at a reduced level compared to target cells including activated microglial cells in the CNS. In this way, by-products and other side effects associated with the compositions are reduced.

Administration of the compositions leads to an improvement, or enhancement, of neurological function in an individual with neurological impairment, or neuronal decline or impairment. In some embodiments, the long-acting GLP-1R agonists are administered to a subject in a therapeutically effective amount to stimulate or induce neural mitosis leading to the generation of new neurons, providing a neurogenic effect. Also provided are effective amounts of the compositions to prevent, reduce, or terminate deterioration, impairment, or death of an individual's neurons, neurites and neural networks, providing a neuroprotective effect.

The actual effective amounts of long-acting GLP-1R agonists can vary according to factors including the specific agent administered, the particular composition formulated, the mode of administration, and the age, weight, condition of the subject being treated, as well as the route of administration and the disease or disorder. In some embodiments, the dose of the long-acting GLP-1R agonist, or pharmaceutical formulation thereof can be from about 0.01 to about 100 mg/kg body weight, from about 0.01 mg/kg to about 10 mg/kg, and from about 0.1 mg to about 5 mg/kg body weight. In other embodiments, the dosage is an absolute amount of a P-GLP-1R agonist, or pharmaceutical formulation thereof, for a single administration to a subject, such as from about 0.1 mg up to about 100 mg. For example, in some embodiments, the dosage of a P-GLP-1R agonist, or pharmaceutical formulation thereof is 0.1 mg, 0.5 mg, 1 mg, 2 mg, 3 mg, 4 mg, 5 mg, 6 mg, 7 mg, 8 mg, 9 mg or 10 mg, or more than 10 mg, for example 20 mg. 30 mg, 40 mg or 50 mg. In an exemplary embodiment, the dosage of P-GLP-1R agonist is 5 mg, administered once a week. Generally, for intravenous injection or infusion, the dosage may be lower than for oral administration.

In a preferred embodiment, the P-GLP-1R agonist is administered in an amount between about 2.5 mg and about 20 mg, inclusive. For example, in a particular embodiment, PEGylated exenatide is administered to a subject in an amount between about 2.5 mg and about 20 mg, inclusive.

In general, the timing and frequency of administration will be adjusted to balance the efficacy of a given treatment schedule with the side-effects of the given delivery system. Exemplary dosing frequencies include continuous infusion, single and multiple administrations such as hourly, daily, weekly, monthly, or yearly dosing.

The long-acting GLP-1R agonist, or pharmaceutical formulation thereof can be administered daily, biweekly, weekly, every two weeks or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more doses daily, once daily, twice weekly, once a week, or less frequently. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀s found to be effective in in vitro and in vivo animal models.

In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

In a preferred embodiment, the P-GLP-1R agonist is administered once weekly in an amount between about 2.5 mg and about 20 mg, inclusive, for a period of between one and twenty weeks, months, or years, inclusive. For example, in a particular embodiment, PEGylated exenatide is administered to a subject once weekly in an amount between about 2.5 mg and about 20 mg, inclusive, for a period of between one and twenty weeks, months, or years, inclusive.

In some embodiments, the amount of a long-lasting GLP-1R agonist administered to a subject changes over time following an initial dose. For example, in some embodiments, the dose of the long-lasting GLP-1R agonist is titrated with weekly increases from about 2.5 mg up to about 20 mg, inclusive.

Therefore, in some embodiments, the amount of PEGylated exenatide administered to a subject changes over time following an initial dose. For example, in some embodiments, the dose of PEGylated exenatide is titrated with weekly increases from about 2.5 mg up to about 20 mg, inclusive.

### C. Combination Therapies and Procedures

The compositions can be administered alone or in combination with one or more conventional therapies. Examples of preferred additional therapeutic agents include other conventional therapies known in the art for treating the desired disease, disorder, or condition.

In the context of chronic impairment associated with SARS-COV-2 infection, the other therapeutic agents can include one or more of anti-infectives, vaccines, anti-inflammatoires, neuroprotective agents, and immune-modulating agents.

Exemplary neuroprotective agents include, for example, glutamate antagonists, antioxidants, and NMDA receptor stimulants. Other neuroprotective agents and treatments include caspase inhibitors, trophic factors, anti-protein aggregation agents, and therapeutic hypothermia.

Other agents for treating neurological dysfunction include amantadine and anticholinergics for treating motor symptoms, clozapine for treating psychosis, cholinesterase inhibitors for treating dementia, and modafinil for treating daytime sleepiness.

### D. Controls

The therapeutic result of the long-acting GLP-1R agonist, or pharmaceutical formulation thereof can be compared to a control or reference. The terms “control” or “reference” refer to a standard of comparison. The term “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result.

uitable controls are known in the art and include, for example, an untreated subject or untreated cells or the same individual prior to treatment.

### V. Kits

The compositions can be packaged in kit. The kit can include a single dose or a plurality of doses of a composition including one or more of the long-acting GLP-1R agonist, or pharmaceutical formulation thereof, and instructions for administering the compositions. Specifically, the instructions direct that an effective amount of the composition be administered to an individual with a particular symptoms, neurological disease, defect, or impairment as indicated. The composition can be formulated as described above with reference to a particular treatment method and can be packaged in any convenient manner.

The present invention will be further understood by reference to the following non-limiting examples.

### EXAMPLES

### Example 1: Expression of GLP-1R on Microglia

To establish whether GLP-1R is expressed on brain-derived microglia and neurons, Western blot analysis of the expression of proteins on primary microglial and neuronal cultured cells was carried out, using Tuj1 and Iba1 as a marker for neuron and microglia, respectively.

It was shown that GLP-1R is expressed on brain-derived microglia and there is little to no expression on neurons.

### Example 2: Production of Pro-Inflammatory Cytokines is Substantially Reduced in the Presence of NLY01 In Vitro

### Methods

Production of pro-inflammatory cytokines is a marker for microglial activation. Chronic microglial activation is pathogenic. To establish the effects of GLP-1R agonists on microglial activation, primary microglia were treated in vitro with PBS or NLY01 and activated or not activated. Cytokine levels were determined using ELISA. Bars indicate mean±s.e.m. (n=3).

### Results

Results are set forth in FIGS. 1A-1D. Briefly, where activated microglia produce pro-inflammatory cytokines in vitro, levels are substantially reduced in the presence of NLY01. In absence of activation, cytokine production by microglia is low and not significantly affected by NLY01 treatment.

### Example 3: NLY01 Inhibits Microglial Activation In Vivo

### Methods

To determine the efficacy of NLY01 on microglial activation in vivo, three-month old C57BL6 mice (male and female) were given a unilateral intrastriatal stereotaxic injection of recombinant α-synuclein PFFs (5 μg/uL) to stimulate microglial activation or PBS. After one month, NLY01 or PBS was delivered subcutaneously (3 mg/kg, twice a week) for 5 months. Mice were perfused and brain tissues collected to allow analysis of activated microglia and cytokine levels following treatment.

### Results

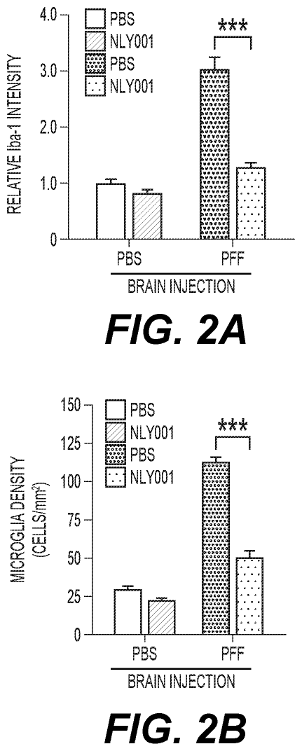

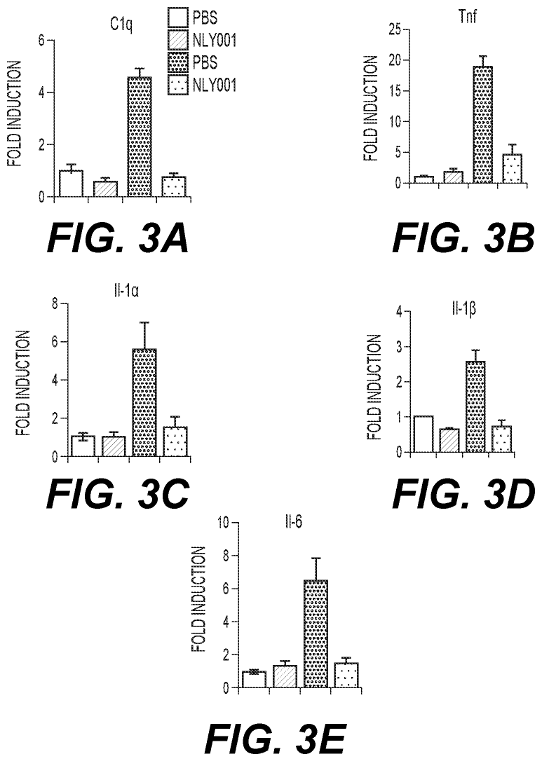

Injection of mouse brain with aggregates of α-synuclein caused microglial activation, production of pro-inflammatory cytokines, and reduced neural function. The effect of NLY01 on microglia in the brain is shown in FIGS. 2A-2B. Iba-1+ activated microglia are elevated after injection with PFFs (black) and this effect is reduced by systemic treatment of the mice with NLY01. Reduced pro-inflammatory cytokine production is apparent in these same mice (FIGS. 3A-3E).

### CONCLUSIONS

NLY01 is a clinically safe, long-acting, GLP-1R agonist with potent effects on microglial activation in vitro and in vivo. NLY01 inhibits microglial activation and improves function in neurodegenerative models. Chronic activation of microglia is associated with neurodegeneration that is regional, depending on the location of the activator. This process plays a role in neurodegenerative disorders such as AD, PD, and ALS. Inhibitors of microglial activation could preserve neural function, thereby slowing the progressive decline in function associated with these chronic and debilitating diseases. All treatments proven clinically effective and approved for treatment of these diseases do not slow progression, they limit symptoms and while the disease continues to progress, effectiveness is lost. NLY01 is different, providing the potential for reduced symptoms and a slowed progression of the disease.

Because microglia are involved in normal adult memory function and activation of microglia is associated with cognitive deficits independent of neurodegeneration, NLY01 and other comparable agents should be effective to treat and/or prevent microglia-dependent brain disorders or symptoms thereof regardless of the activator and may affect both short and long-term memory functions. Inhibition of microglial activation by NLY01 reduces astrocyte formation and both microglia and astrocytes are involved in memory. Activated microglia have a reduced capacity to participate in normal memory function and are associated with reduced cognitive ability. A wide variety of viral infections can lead to neurologic dysfunction. In the case of COVID-19, persistent microglial activation is associated with cognitive deficits.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A method of treating or preventing neurological impairment induced by, or resulting from, infection with a virus in a subject, comprising
administering to the subject a pharmaceutically effective amount of a composition comprising a long-acting GLP-1R agonist to reduce microglial activation.

2. The method of claim 1, wherein the long-acting GLP-1R agonist comprises a PEGylated GLP-1r agonist analog, a Fc fusion GLP-1 agonist analog, an albumin fusion GLP-1 analog, or derivatives thereof.

3. The method of claim 2, wherein the long-acting GLP-1R agonist comprises a PEGylated exenatide or a PEGylated exenatide analog.

4. The method of claim 1, wherein the amount of the long-acting GLP-1r agonist is effective to inhibit the secretion of inflammatory and/or neurotoxic mediators secreted from activated microglial cells.

5. The method of claim 4, wherein secretion of inflammatory and/or neurotoxic mediators by astrocytes is also inhibited.

6. The method of claim 4, wherein the long-acting GLP-1r agonist is in an effective amount to reduce inflammatory or neurotoxic mediators selected from the group consisting of TNF-α, IL-1α, IL-1β, IFN-γ, IL-6, and C1q as compared to an appropriate control.

7. The method of claim 1, wherein the effective amount of the long-acting GLP-1r agonist reduces the cell populations of activated microglia in the subject.

8. The method of claim 1, wherein the GLP-1R agonist is effective to reduce one or more symptoms of fatigue, cognitive difficulties, neurological problems, headache, and/or numbness/tingling.

9. The method of claim 8, wherein the cognitive difficulties comprise impairment of one or more functions selected from the group consisting of reasoning, problem solving, spatial planning, target detection, motor skills, and memory, as compared to the same function in the same subject prior to infection with the virus.

10. The method of claim 1, wherein one or more symptoms of neurological impairment are selected from the group consisting of low energy, fatigue, and cognitive difficulties, such as problems concentrating, disorientation and difficulty finding the right words, confusion, inattention, and memory loss.

11. The method of claim 8, wherein the subject has no other symptoms associated with the infection.

12. The method of claim 1, wherein the subject has not been diagnosed with a neurodegenerative disease or disorder prior to the infection.

13. The method of claim 1, wherein the virus is selected from the group consisting of SARS-COV-2 virus, Rabies virus, Japanese encephalitis virus, Dengue virus, West Nile virus (WNV), and Zika virus.

14. The method of claim 13, wherein the virus is a SARS-COV-2 virus.

15. The method of claim 14, wherein the subject was infected with the SARS-COV-2 virus one, two, three, four, five, six, seven, eight, none, ten or more than ten days, weeks, or months prior to the onset of one or more symptom of neurological impairment.

16. The method of claim 1, wherein the composition is administered via a route selected from the group consisting of oral administration, intravenous administration, parenteral administration, and subcutaneous administration.

17. The method of claim 1, wherein the composition is administered in a form selected from the group consisting of pills, capsules, tablets, liquids, and suspensions

18. The method of claim 1, wherein the composition is administered to the subject between 1 and 12 times in a month, inclusive.

19. The method of claim 1, wherein the composition is administered at an interval selected from the group consisting of once a week, once every two weeks, approximately once a month, once every two months and once every three months.

20. The method of claim 1, wherein the composition is administered once a week for a period of 12 weeks.

21. The method of claim 1, wherein the composition has a half-life in vivo of 12±8 days inclusive in non-human primates or humans.

22. The method of claim 1, wherein the composition is administered to a human subject at a dose of between 0.001 mg/kg body weight of the subject and 100 mg/kg body weight of the subject, inclusive.

23. The method of claim 3, wherein the composition is administered to a human subject at a dose of between 2.0 mg and 20 mg, inclusive.

24. The method of claim 23, wherein the composition is administered to the subject at a dose of 5 mg.

25. The method of claim 24, wherein the composition is administered to the subject once a week for up to 6 months.

26. The method of claim 1, wherein the composition is administered to the subject for a duration of between one and 10 days, weeks, months, or years, inclusive.

27. The method of claim 1, wherein the methods reduce pathological microglial activation associated with COVID-19.

28. The method of claim 1, wherein the subject has Alzheimer's disease or Parkinson's disease.

29. The method of claim 1 where the subject has fibromyalgia.
